(19) United States
(12) Patent Application Publication
(10) Pub. No.: US 2008/0166308 A1
(43) Pub. Date: Jul. 10, 2008

(54) Tanning Method

(76) Inventors: Frank Pfluecker, Darmstadt (DE); Sabine Hitzel, Darmstadt (DE); Hansjuergen Driller, Gross-Umstadt (DE); Silke Hornung, Roedermark (DE); Heike Schuchmann, Rossdorf (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/997,629

(22) PCT Filed: Jul. 5, 2006
(86) PCT No.: PCT/EP2006/006555

§ 371 Date: Feb. 1, 2008

(30) Foreign Application Priority Data
Aug. 2, 2005 (DE) 10 2005 036 793.3

(51) Int. Cl. A61K 8/34; A61Q 17/04

(52) U.S. Cl. 424/59

(57) ABSTRACT

The present invention relates to a method for tanning the human body and in particular for avoiding application edges during tanning, which is characterised in that at least one formulation comprising 0.1 to 1% by weight of at least one self-tanning agent substance is applied repeatedly, but at least once daily, to the human body or parts of the human body, and to a cosmetic formulation which is suitable for the said purpose.

The present invention relates to a method for tanning the human body and in particular for avoiding application edges during tanning, and to a cosmetic formulation which is suitable for the said purpose.

In particular in regions in which pale skin types predominate, tanning of the human skin is regarded as a sign of wellbeing and health.

However, natural tanning by the UV radiation present in sunlight also entails risks, such as premature skin ageing or also an increased risk of suffering from skin cancer.

In order to reduce this risk, on the one hand UV filter compositions which are said to filter the harmful components of UV radiation are offered.

By contrast, so-called “pre-tan products” or “tan promoters”, which have to be applied before exposure to the sun, are also offered. Yellowing of these compositions then occurs in the sun, which is intended to result in a slight brown-yellow coloration of the epidermis, which additionally enhances the “sun an” and thus shortens the time for which the body has to be exposed to the sun.

A further type of artificial tanning which is completely independent of UV light can be caused by the hormones that are usually liberated in the body, also as a consequence of (natural) UV exposure, and which ultimately stimulate melanocytes to synthesise melanin. Mention may be made in this connection of, for example, derivatives of propiomelanocortin (POMC), such as aMSH and synthetic variants (such as NDP), some of which have much higher activity than natural aMSH. Although tanning can in principle be caused by these hormones, the use thereof in cosmetics is ruled out since they are clearly substances with a pharmacological action (hormones) which should not be employed widely without medical indication.

Tinting of the skin by self-tanning agents also takes place entirely without exposure to sunlight. Thus, U.S. Pat. No. 5,514,367 discloses self-tanning agent compositions which can comprise 0.1 to 20% by weight of dihydroxyacetone. U.S. Pat. No. 5,858,334 discloses self-tanning agent compositions which can comprise 0.5 to 10% by weight of dihydroxyacetone. US 2004/0185072 discloses care products which comprise ectoin and dihydroxyacetone. U.S. Pat. No. 6,451,293 discloses that the combination of dihydroxyacetone with erythrulose gives particularly good tanning results.

However, a problem on use of self-tanning agents is uniform application to the human skin in an adequately high active-compound concentration. In addition, so-called application edges occur on application, in particular, on the face or at other edges of the application zone. These application edges form, for example, since creams are only applied to just up to the hairline and tanning takes place up to this application edge.

It has also been proposed to apply self-tanning agent solutions by means of active-compound showers. However, large amounts of self-tanning agents are necessary here, and uniform application can likewise only be ensured to a limited extent.

Surprisingly, it has now been found that the requisite active-compound concentration can be reduced if the application of the self-tanning agents is carried out repeatedly.

The present invention therefore relates firstly to a method for tanning the human body, which is characterised in that at least one formulation comprising 0.1 to 1% by weight of at least one self-tanning agent substance is applied repeatedly, but at least once daily, to the human body or parts of the human body.

In a preferred variant of the invention, the repeated application serves to avoid application edges. The present invention therefore furthermore relates to a method for avoiding application edges during tanning of the human body, which is characterised in that at least one formulation comprising 0.1 to 1% by weight of at least one self-tanning agent substance is applied repeatedly, but at least once daily, to the human body or parts of the human body. This use utilises the effect that, on repeated manual application, the application edges are never actually identical, but usually differ from one another by at least a few millimetres. Slow “blending” of the tan to the untreated skin shade is thus achieved at the application edges.

It has proven advantageous here for the at least one formulation to be applied twice or three times daily, preferably twice daily.

In a variant of the invention, the application is carried out in the morning by means of a day-care formulation comprising 0.1 to 1% by weight of at least one self-tanning agent substance and in the evening by means of a night-care formulation comprising 0.1 to 1% by weight of at least one self-tanning agent substance.

The present invention furthermore relates to cosmetic formulations which are particularly suitable for the use according to the invention. Cosmetic formulations comprising at least one self-tanning substance, characterised in that the formulation comprises more than 0.1% by weight and less than 0.5% by weight of the at least one self-tanning agent substance, are therefore claimed.

For the purposes of the present invention, self-tanning substances or self-tanning agents are taken to mean all substances or substance mixtures which are capable of tanning human skin without exposure to UV radiation. Advantageous self-tanning agents which can be employed for the purposes of the present invention are the following substances:

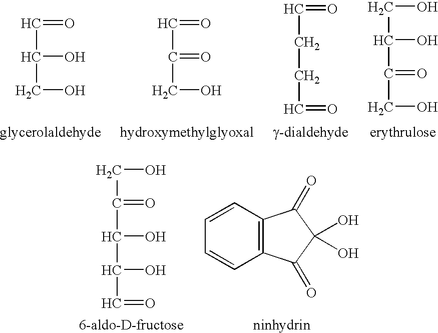

Mention may furthermore be made of 5-hydroxy-1,4-naphthoquinone (juglone), which is extracted from the shells of fresh walnuts,

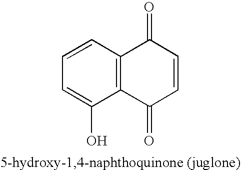

and 2-hydroxy-1,4-naphthoquinone (lawsone), which occurs in henna leaves.

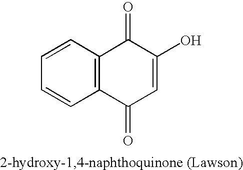

The most important active compound for self-tanning in accordance with the present invention is 1,3-dihydroxyacetone (DHA), a trifunctional sugar which occurs in the human body.

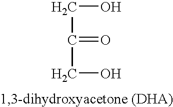

The preferred concentration in accordance with the invention of the at least one self-tanning substance is in the range from 0.1 to 1% by weight, it being particularly preferred for the formulation to comprise more than 0.1% by weight and less than 0.5% by weight of the self-tanning substance. It may be particularly preferred in accordance with the invention to employ mixtures of self-tanning substances. It is especially preferred here for the formulation according to the invention to comprise essentially dihydroxyacetone or erythrulose or a mixture thereof as self-tanning agent.

The skin tan achieved in the manner according to the invention cannot be washed off and is retained evenly in the case of regular application, whereas in the case of the previously known once-only self-tanning agent treatment, the tan disappears with the normal shedding of the skin (after about 10-15 days). In addition, the multiple application means that a particularly homogeneous distribution of the self-tanning agents and thus of the tan is also achieved. The low self-tanning agent dosage in accordance with the invention additionally means that the tan—in a similar way to suntan—is only built up slowly and odour nuisances and eye irritations are completely or at least substantially suppressed. In addition, skin tanned using self-tanning agents in the manner according to the invention exhibits permanently improved UV-A protection, which can be determined, for example, by means of the modified PPD protocol of the Japan Cosmetic Industry Association (1995) via the b* value of the L-a-b system.

Addition of hydrophilic solvents enables the intensity of the tan to be additionally increased. This makes it possible to reduce the concentration of the self-tanning substance further. Furthermore, the hydrophilic solvents can ensure more uniform distribution of the self-tanning substance.

The hydrophilic solvents to be employed in accordance with the invention can advantageously be selected from the following substance groups:
monoalcohols having a low C number, for example isopropanol,
polyfunctional alcohols, such as, preferably, propylene glycol or glycerol,
esters of fatty alcohols with alkanoic acids having a low C number.

The hydrophilic solvents which are preferred in accordance with the invention are propylene glycol and/or glycerol.

The preferred concentration of hydrophilic solvents, in particular propylene glycol and/or glycerol, in formulations according to the invention is in the range from 0.1 to 20 percent by weight.

In addition, the presence of ceramides, cholesterol, phospholipids, cholesteryl sulfate, cholesteryl phosphate, phosphatidylcholine, lecithin and/or empty liposomes may result in increased tanning intensity.

These substances are generally also known as “entrainers” since they transport the self-tanning agent molecules to deeper layers of the stratum corneum.

In accordance with the invention, phospholipids are taken to mean the following substances: phosphatidic acids, the actual lecithins, cardolipins, lysophospholipids, lysolecithins, plasmalogens, phosphosphingolipids, sphingomyelins. Preferred substances are described below.

Phosphatidic acids are glycerol derivatives which are esterified by means of fatty acids in the 1-sn- and 2-position (1-sn-position: usually saturated, 2-position: usually mono- or polyunsaturated), but esterified by means of phosphoric acid at atom 3-sn and are characterised by the general structural formula

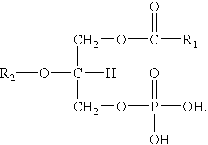

In the phosphatidic acids which occur in human or animal tissue, the phosphate radical is usually esterified by means of amino alcohols, such as choline (lecithin=3-sn-phosphatidylcholine) or 2-aminoethanol (ethanolamine) or L-serine (cephalin=3-sn-phosphatidylethanolamine or sn-phosphatidyl-L-serine), by means of myo-inositol to give the phosphoinositides which are common in tissues [1-(3-sn-phosphatidyl)-D-myoinositols], by means of glycerol to give phosphatidylglycerols. Particular preference is given to lecithins (=3-sn-phosphatidylcholine).

Lecithins are characterised by the general structural formula

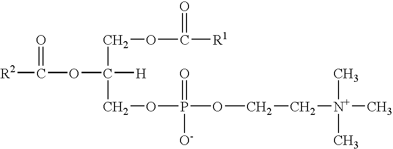

where R¹ and R² typically represent unbranched aliphatic radicals having 15 or 17 carbon atoms and up to 4 cis double bonds.

Cardiolipins (1,3-bisphosphatidylglycerols) are phospholipids made from two phosphatidic acids linked via glycerol.

Lysophospholipids are obtained if an acyl radical is cleaved off from phospholipids by means of phospholipase A (for example lysolecithins). Lysophospholipids are characterised by the general structural formula

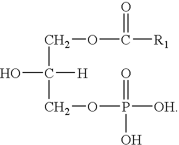

Lysolecithins, for example, are characterised by the general structural formula

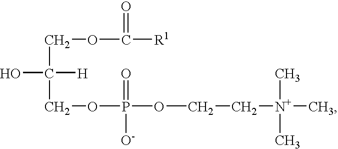

where R¹ typically represents unbranched aliphatic radicals having 15 or 17 carbon atoms and up to 4 cis double bonds.

The phospholipids also include plasmalogens, in which an aldehyde (in the form of an enol ether) is bonded instead of a fatty acid in the 1-position; the O-1-sn-alkenyl compounds which correspond to the phosphatidylcholines are called, for example.

The basic structure on which phosphosphingolipids are based is sphingosine or also phytosphingosine, which are distinguished by the following structural formulae:

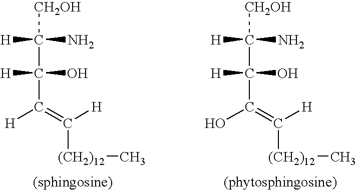

Modifications of sphingolipids are distinguished, for example, by the general basic structure:

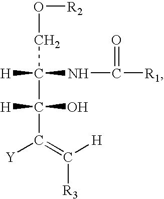

in which R₁ and R₃ represent, independently of one another, saturated or unsaturated, branched or unbranched alkyl radicals having 1 to 28 carbon atoms, R₂ is selected from the group: hydrogen atom, saturated or unsaturated, branched or unbranched alkyl radicals having 1 to 28 carbon atoms, sugar radicals, phosphate groups, which have been esterified by means of organic radicals or are unesterified, sulfate groups which have been esterified by means of organic radicals or are unesterified, and Y represents either a hydrogen atom, a hydroxyl group or another heterofunctional radical.

### Sphingophospholipids

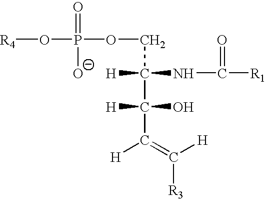

in which R₁ and R₃ represent alkyl radicals, R₄ represents an organyl radical.

Sphingomyelins are organylphosphorylated sphingolipids of the

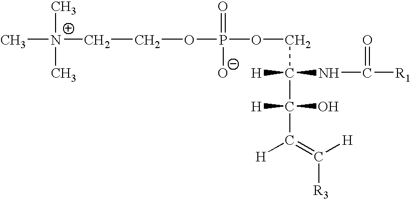

type.

Particularly preferred phospholipids are lecithins. Lecithin products which are advantageous to use are selected from crude lecithins which have been deoiled and/or fractionated and/or spray-dried and/or acetylated and/or hydrolysed and/or hydrogenated. They are commercially available. Preference is given to soya lecithins.

Phospholipids which are advantageous to use in accordance with the invention are, for example, commercially available under the trade names Phospholipon 25 or Phospholipon 90 (Nattermann), Emulmetik 120 (Lucas Meyer), Sternpur E (Stern), Sternpur PM (Stern), Nathin 3KE (Stern), Phospholipon 90H (Nattermann/Rhone-Poulenc), Lipoid S 100 (Lipoid).

The preferred concentration of entrainers of this type is, in accordance with the invention, in the range from 0.1 to 10 percent by weight.

Under the influence of ultraviolet radiation, DHA can eliminate formaldehyde in small amounts. It is therefore preferred in accordance with the invention if the formulations comprise UV filters for stabilisation. Since these UV filters also come into contact with the skin on application of the formulation, the UV filters should be tolerated when applied topically. The additional advantage arises here that these UV filters are likewise absorbed uniformly into the skin on application and thus protect the skin against UV radiation.

Particular preference is given to UV filters whose physiological acceptability has already been demonstrated. Both for UV-A and UV-B filters, there are substances known from the specialist literature, for example

benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxo-born-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of 0.5 to 10 percent by weight, preferably 1-8%.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB), dimethicone diethylbenzalmalonate (CAS No. 207 574-74-1)
2,2′-methylenebis(6-(2H-benzotriazol-2-yl)-4-(1,1,3,3-tetramethylbutyl)-phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxyl]phenyl}-6-(4-methoxyphenyl)-1,3,5-triazine (CAS No. 103 597-45-, 187 393-00-6),
2-ethyl hexyl 4,4′-[(6-[4-((1,1-dimethylethyl)aminocarbonyl)phenylamino]-1,3,5-triazine-2,4-diyl)diimino]bis(benzoate) (for example Uvasorb® HEB).

Further suitable UV filters are also methoxyflavones corresponding to German patent application DE 10232595.

Organic UV filters are generally incorporated into cosmetic formulations in an amount of 0.5 to 20 percent by weight, preferably 1-15%.

Conceivable inorganic UV filters are those from the group of the titanium dioxides, such as, for example, coated titanium dioxide (for example Eusolex® T-2000, Eusolex® T-AQUA), zinc oxides (for example Sachtotec®), iron oxides or also cerium oxides. These inorganic UV filters are generally incorporated into cosmetic compositions in an amount of 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding sub-stances which is proposed here suppresses this effect.

In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions according to the invention are preferably present in amounts which ensure that the encapsulated UV filters are pre-sent in the composition in the above-indicated amounts.

The compositions according to the invention may in addition comprise further conventional skin-protecting or skin-care active compounds. These can in principle be any active compounds known to the person skilled in the art.

These may be chromone derivatives. The term chromone derivatives here is preferably taken to mean certain chromen-2-one derivatives which are suitable as active compounds for the preventive treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula I

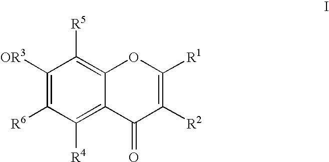

where

R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,

R³ stands for H or straight-chain or branched C₁- to C₂₀-alkyl groups,

R⁴ stands for H or OR⁸,

R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom in the chain and furthermore the alkyl chain may also be interrupted by oxygen, and

R⁷ stands for H, straight-chain or branched C₁- to C₂₀-alkyl groups, a polyhydroxyl compound, such as preferably an ascorbic acid radical or glycosidic radicals, and

R⁸ stands for H or straight-chain or branched C₁- to C₂₀-alkyl groups, where at least 2 of the substituents R¹, R², R⁴-R⁶ are not H or at least one substituent from R¹ and R² stands for —C(═O)—R⁷ or —C(═O)—OR⁷.

The proportion of one or more compounds selected from chromone derivatives in the composition according to the invention is preferably 0.001 to 5% by weight, particularly preferably 0.01 to 2% by weight, based on the composition as a whole.

A protective action against oxidative stress or against the effect of free radicals of the formulations according to the invention can be achieved if the compositions comprise one or more antioxidants, the person skilled in the art being presented with absolutely no difficulties in selecting antioxidants which act suitably quickly or in a delayed manner.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Anti-oxidants of this type are usually employed with compounds of the formula I in such compositions in ratios in the range from 1000:1 to 1:1000, preferably in amounts of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology&Medicine 2001, 31(7), 869-881, are investigating the pH dependence of the antioxidant action of hydroxyflavones. Quercetin exhibits the highest activity amongst the structures investigated over the entire pH range.

Suitable antioxidants are furthermore compounds of the formula II

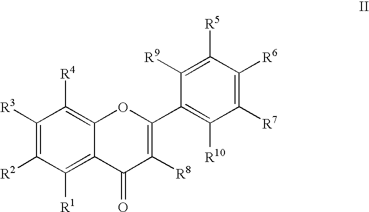

where R¹ to R¹⁰ may be identical or different and are selected from
   H
   OR¹¹
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom in the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ— groups, where n=1 to 3,
   where all OR¹¹, independently of one another, stand for
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to a primary or secondary carbon atom in the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or mono- and/or oligoglycosyl radicals,
   with the proviso that at least 4 radicals from R¹ to R⁷ stand for OH and that at least 2 pairs of adjacent —OH groups are present in the molecule,
   or R², R⁵ and R⁶ stand for OH and the radicals R¹, R³, R⁴ and R⁷⁻¹⁰ stand for H,
      as described in the earlier German patent application DE 10244282.7.

Particularly preferred active compounds are also pyrimidinecarboxylic acids and/or aryl oximes.

Pyrimidinecarboxylic acids occur in halophilic microorganisms and play a role in the osmoregulation of these organisms (E. A. Galinski et al., Eur. J. Biochem., 149 (1985) pages 135-139). Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacterial cells. Pharmaceutical products, such as non-glycosylated, pharmaceutically active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European patent application EP-A-0 671 161 describes, in particular, that ectoin and hydroxyectoin are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula III

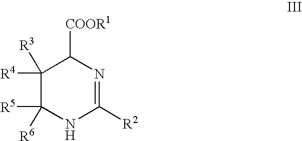

in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and skin appendages. The compositions here preferably comprise 0.01 to 10% by weight of the aryl oxime, it being particularly preferred for the composition to comprise 0.05 to 5% by weight of aryl oxime.

Furthermore, the compositions according to the invention may also comprise dyes and coloured pigments. The dyes and coloured pigments can be selected from the corresponding positive list in the German Cosmetics Regulation or the EC list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | Green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-Chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′-sulfonyldiethylamide-1′-phenylazo)-3- | 12490 | Red |
| hydroxy-5″-chloro-2″,4″-dimethoxy-2-naphthanilide | | |
| Disperse Yellow 16 | 12700 | Yellow |
| 1-(4-Sulfo-1-phenylazo)-4-aminobenzene-5-sulfonic acid | 13015 | Yellow |
| 2,4-Dihydroxyazobenzene-4′-sulfonic acid | 14270 | Orange |
| 2-(2,4-Dimethylphenylazo-5-sulfonyl)-1-hydroxynaphthalene- | 14700 | Red |
| 4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic | 15850 | Red |
| acid | | |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3-carboxylic | 15880 | Red |
| acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo-1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo-7′-sulfo-1′-naphthylazo]-1- | 27755 | Black |
| hydroxy-7-aminonaphthalene-3,6-disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″- | 42051 | Blue |
| bis(diethylamino)triphenylcarbinol | | |
| 4-[(-4-N-Ethyl-p-sulfobenzylamino)phenyl-(4-hydroxy-2- | 42053 | Green |
| sulfophenyl)(methylene)-1-(N-ethyl-N-p-sulfobenzyl)-2,5- | | |
| cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)Δ^{2,5}-cyclohexadienimine | | |
| Acid Green 9 | 42100 | Green |
| Diethyldisulfobenzyldi-4-amino-2-chlorodi-2-methylfuchsonimmonium | 42170 | Green |
| Basic Violet 14 | 42510 | Violet |
| Basic Violet 2 | 42520 | Violet |
| 2′-Methyl-4′-(N-ethyl-N-m-sulfobenzyl)amino-4″-(N- | 42735 | Blue |
| diethyl)amino-2-methyl-N-ethyl-N-m-sulfobenzylfuchsonimmonium | | |
| 4′-(N-Dimethyl)amino-4″-(N-phenyl)aminonaphtho-N-di- | 44045 | Blue |
| methylfuchsonimmonium | | |
| 2-Hydroxy-3,6-disulfo-4,4′-bisdimethylaminonaphtho- | 44090 | Green |
| fuchsonimmonium | | |
| Acid Red 52 | 45100 | Red |
| 3-(2′-Methylphenylamino)-6-(2′-methyl-4′-sulfophenyl- | 45190 | Violet |
| amino)-9-(2″-carboxyphenyl)xanthenium salt | | |
| Acid Red 50 | 45220 | Red |
| Phenyl-2-oxyfluorone-2-carboxylic acid | 45350 | Yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium/aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | Orange |
| Indigo | 73000 | Blue |
| Indigodisulfonic acid | 73015 | Blue |
| 4,4′-Dimethyl-6,6′-dichlorothioindigo | 73360 | Red |
| 5,5′-Dichloro-7,7′-dimethylthioindigo | 73385 | Violet |
| Quinacridone Violet 19 | 73900 | Violet |
| Pigment Red 122 | 73915 | Red |
| Pigment Blue 16 | 74100 | Blue |
| Phthalocyanines | 74160 | Blue |
| Direct Blue 86 | 74180 | Blue |
| Chlorinated phthalocyanines | 74260 | Green |
| Natural Yellow 6, 19; Natural Red 1 | 75100 | Yellow |
| Bixin, Nor-Bixin | 75120 | Orange |
| Lycopene | 75125 | Yellow |
| trans-alpha-, -beta- or -gamma-Carotene | 75130 | Orange |
| Keto and/or hydroxyl derivatives of carotene | 75135 | Yellow |
| Guanine or pearlescent agent | 75170 | White |
| 1,7-Bis(4-hydroxy-3-methoxyphenyl)-1,6-heptadiene-3,5- | 75300 | Yellow |
| dione | | |
| Complex salt (Na, Al, Ca) of carminic acid | 75470 | Red |
| Chlorophyll a and b; copper compounds of chlorophylls | 75810 | Green |
| and chlorophyllines | | |
| Aluminium | 77000 | White |
| Aluminium hydroxide | 77002 | White |
| Water-containing aluminium silicates | 77004 | White |
| Ultramarine | 77007 | Blue |
| Pigment Red 101 and 102 | 77015 | Red |
| Barium sulfate | 77120 | White |
| Bismuth oxychloride and mixtures thereof with mica | 77163 | White |
| Calcium carbonate | 77220 | White |
| Calcium sulfate | 77231 | White |
| Carbon | 77266 | Black |
| Pigment Black 9 | 77267 | Black |
| Carbo medicinalis vegetabilis | 77268:1 | Black |
| Chromium oxide | 77288 | Green |
| Chromium oxide, water-containing | 77278 | Green |
| Pigment Blue 28, Pigment Green 14 | 77346 | Green |
| Pigment Metal 2 | 77400 | Brown |
| Gold | 77480 | Brown |
| Iron oxides and hydroxides | 77489 | Orange |
| Iron oxide | 77491 | Red |
| Iron oxide hydrate | 77492 | Yellow |
| Iron oxide | 77499 | Black |
| Mixtures of iron(II) and iron(III) hexacyanoferrate | 77510 | Blue |
| Pigment White 18 | 77713 | White |
| Manganese ammonium diphosphate | 77742 | Violet |
| Manganese phosphate; Mn₃(PO₄)₂•7H₂O | 77745 | Red |
| Silver | 77820 | White |
| Titanium dioxide and mixtures thereof with mica | 77891 | White |
| Zinc oxide | 77947 | White |
| 6,7-Dimethyl-9-(1′-D-ribityl)isoalloxazine, lactoflavin | | Yellow |
| Sugar dye | | Brown |
| Capsanthin, capsorubin | | Orange |
| Betanin | | Red |
| Benzopyrylium salts, anthocyans | | Red |
| Aluminium, zinc, magnesium and calcium stearate | | White |
| Bromothymol Blue | | Blue |

It may furthermore be favourable to select, as dye, one or more substances from the following group:

2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:
1. Natural pearlescent pigments, such as, for example, “pearl essence” (guanine/hypoxanthine mixed crystals from fish scales) and “mother-of-pearl” (ground mussel shells)
2. Monocrystalline pearlescent pigments, such as, for example, bismuth oxychloride (BiOCl)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| Group | thickness | Colour |
| Silver-white pearlescent | TiO₂: 40-60 nm | Silver |
| pigments | | |
| Interference pigments | TiO₂: 60-80 nm | Yellow |
| | TiO₂: 80-100 nm | Red |
| | TiO₂: 100-140 nm | Blue |
| | TiO₂: 120-160 nm | Green |
| Coloured lustre pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold shades |
| | TiO₂/Cr₂O₃ | Green |
| | TiO₂/Berlin Blue | Dark blue |

Particular preference is given to, for example, the pearlescent pigments available from Merck under the trade names Timiron, Colorona or Dichrona.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂- and Fe₂O₃-coated SiO₂ particles (“Ronasphere” grades), which are marketed by Merck and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks. Due to their particle size of 40-80 μm, they have a glitter effect in addition to the colour.

Also particularly advantageous are effect pigments available from Flora Tech in various colours (yellow, red, green, blue) under the trade name Metasomes Standard/Glitter. The glitter particles here are in the form of mixtures with various assistants and dyes (such as, for example, the dyes having the Colour Index (CI) numbers 19140, 77007, 77289, 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably 0.5 to 15% by weight, in particular 1.0 to 10% by weight, in each case based on the total weight of the compositions.

All compounds or components which can be used in the compositions are either known and commercially available or can be synthesised by known processes.

The compositions according to the invention may in addition comprise further conventional skin-protecting or skin-care active compounds. These can in principle be any active compounds known to the person skilled in the art.

In a preferred embodiment of the present invention, the cosmetic formulation can be in the form of a liquid, creamy, milky and/or gelatinous day-care product.

It is furthermore preferred for the self-tanning agent formulations to comprise moisture-donating substances, such as, for example, erythrulose or the above-mentioned ectoins.

In particular, it may also be advantageous for amino acids, oligoamino acids or proteins which react in situ with the self-tanning agent to be used in addition to the self-tanning agent composition. Preferred compounds to be added here are, in particular, lysine, glycine, methionine and methionine sulfoxide. An advantageous dispensing form here is a multiphase composition, which is only mixed on application, for example by the application or dispensing from a multichamber container. An example is a two-phase cream whose first layer comprises the self-tanning agent and whose other layer comprises the amino acids.

The following examples are intended to explain the invention in greater detail without limiting its scope.

For the ingredients, the INCI names (by definition in English) are indicated in each case. In the case of mixtures, the trade name is indicated instead of the INCI name.

### EXAMPLES

### Example 1

### Skin-Care Cream

| | Ingredient | [%] |
|---|---|---|
| A | Tego Care 150; Goldschmidt | 8.00 |
| | CETEARYL ALCOHOL | 1.50 |
| | CETEARYL ETHYLHEXANOATE | 6.50 |
| | CAPRYLIC/CAPRIC TRIGLYCERIDE | 6.50 |
| | STEAROXY DIMETHICONE | 1.20 |
| | DIMETHICONE | 0.50 |
| | PROPYLPARABEN | 0.05 |
| B | PROPYLENE GLYCOL | 3.00 |
| | ECTOIN | 0.30 |
| | METHYLPARABEN | 0.15 |
| | AQUA (WATER) | 61.45 |
| C | DIHYDROXYACETONE | 0.75 |
| | AQUA (WATER) | 10.00 |
| D | Perfume oil | 0.10 |

### Preparation:

Phases A and B are heated separately to 80° C., and phase B is slowly added to phase A with stirring. After homogenisation and cooling, phases C and D are added at 40° C.

### Recommended Application:

Apply twice daily (morning and evening) to the skin areas to be tanned.

### Example 2

### Lotions (W/O)

| | ST-16-34 | ST-16-35 | ST-16-36 | ST-16-37 |
|---|---|---|---|---|
| Ingredient | [%] | [%] | [%] | [%] |
| Phase A | | | | |
| Abil EM 97 | 1.50 | 1.50 | 1.50 | 1.50 |
| Abil EM 90 | 1.30 | 1.30 | 1.30 | 1.30 |
| Cyclomethicone | 13.00 | 13.00 | 13.00 | 13.00 |
| Ethylhexyl Palmitate | 2.00 | 2.00 | 2.00 | 2.00 |
| SFE 839 | 5.00 | 5.00 | 5.00 | 5.00 |
| Diethylhexyl Carbonate | 5.00 | 5.00 | 5.00 | 5.00 |
| Perfume | 0.30 | 0.30 | 0.30 | 0.30 |
| Phase B | | | | |
| Dihydroxyacetone | 0.10 | 0.15 | 0.25 | 0.45 |
| Ectoin | 0.30 | 0.30 | 0.30 | 0.30 |
| Propylene Glycol | 18.00 | 18.00 | 18.00 | 18.00 |
| Glycerin | 3.00 | 3.00 | 3.00 | 3.00 |
| Magnesium Sulfate | 2.00 | 2.00 | 2.00 | 2.00 |
| Alcohol | 8.00 | 8.00 | 8.00 | 8.00 |
| Phenonip | 1.00 | 1.00 | 1.00 | 1.00 |
| Aqua (Water) | 39.50 | 39.45 | 39.35 | 39.15 |
| Total | 100.00 | 100.00 | 100.00 | 100.00 |

| | ST-16-38 | ST-16-39 | ST-16-40 |
|---|---|---|---|
| Ingredient | [%] | [%] | [%] |
| Phase A | | | |
| Abil EM 97 | 1.50 | 1.50 | 1.50 |
| Abil EM 90 | 1.30 | 1.30 | 1.30 |
| Cyclomethicone | 13.00 | 13.00 | 13.00 |
| Ethylhexyl Palmitate | 2.00 | 2.00 | 2.00 |
| SFE 839 | 5.00 | 5.00 | 5.00 |
| Diethylhexyl Carbonate | 5.00 | 5.00 | 5.00 |
| Perfume | 0.30 | 0.30 | 0.30 |
| Phase B | | | |
| Dihydroxyacetone | 0.50 | 0.80 | 0.80 |
| Erythrulose | | 0.50 | 0.50 |
| Ectoin | 0.30 | 0.30 | 0.30 |
| Ethoxydiglucol | | | 1.00 |
| Propylene Glycol | 18.00 | 18.00 | 17.00 |
| Glycerin | 3.00 | 3.00 | 3.00 |
| Magnesium Sulfate | 2.00 | 2.00 | 2.00 |
| Alcohol | 8.00 | 8.00 | 8.00 |
| Phenonip | 1.00 | 1.00 | 1.00 |
| Aqua (Water) | 39.10 | 38.30 | 38.30 |
| Total | 100.00 | 100.00 | 100.00 |

### Preparation:

Magnesium Sulfate Heptahydrate is dissolved in the water of phase B, and the remaining ingredients of phase B are added. Phase B is slowly added to phase A with stirring (MFR mixer at 300-400 rpm). The mixture is stirred at 1200 rpm for 2 minutes and subsequently homogenised at 2000 rpm for 2 minutes.

| Trade name | INCI |
|---|---|
| Abil EM 97 | Bis-PEG/PPG-14/14, Dimethicone, |
| | Cyclopentasiloxane |
| Abil EM 90 | Cetyl PEG/PPG-10/1, Dimethicone |
| SFE 839 | Cyclopentasiloxane, |
| | Dimethicone/Vinyldimethicone Cross- |
| | polymer |
| Phenonip | Phenoxyethanol, Butylparaben, |
| | Ethylparaben, Propylparaben, |
| | Methylparaben |

### Example 3

### Hydrogel

| | SF-40-08 | SF-40-09 | SF-40-10 | SF-40-11 |
|---|---|---|---|---|
| Ingredient | [%] | [%] | [%] | [%] |
| Phase A | | | | |
| Dihydroxyacetone | 0.10 | 0.15 | 0.25 | 0.45 |
| Ectoin | 0.30 | 0.30 | 0.30 | 0.30 |
| Propylene Glycol | 2.50 | 2.50 | 2.50 | 2.50 |
| Sorbitol | 2.50 | 2.50 | 2.50 | 2.50 |
| Methylparaben | 0.2 | 0.2 | 0.2 | 0.2 |
| Aqua (Water) | 32.90 | 32.85 | 32.75 | 32.55 |
| Phase B | | | | |
| Aqua (Water) | 60.00 | 60.00 | 60.00 | 60.00 |
| Hydroxyethylcellulose | 1.50 | 1.50 | 1.50 | 1.50 |
| Total | 100.00 | 100.00 | 100.00 | 100.00 |

### Preparation:

Hydroxyethylcellulose is added to the water of phase B with vigorous stirring. The addition must take place sufficiently slowly that the particles are able to separate and their surfaces are individually wetted, but should also be sufficiently fast to minimise the viscosity of the aqueous phase. Dihydroxyacetone is added to the water of phase A, and the remaining ingredients are added with stirring. Phases A and B are combined and homogenised.

| | SF-40-12 | SF-40-13 | SF-40-14 | SF-40-15 |
|---|---|---|---|---|
| Ingredient | [%] | [%] | [%] | [%] |
| Phase A | | | | |
| Dihydroxyacetone | 0.50 | 0.80 | 0.80 | 0.80 |
| Erythrulose | | | 0.50 | 0.50 |
| Ectoin | 0.30 | 0.30 | 0.30 | 0.30 |
| Ethoxydiglycol | | | | 1.00 |
| Propylene Glycol | 2.50 | 2.50 | 2.50 | 2.00 |
| Sorbitol | 2.50 | 2.50 | 2.50 | 2.00 |
| Methylparaben | 0.2 | 0.2 | 0.2 | 0.2 |
| Aqua (Water) | 32.50 | 32.20 | 31.70 | 31.70 |
| Phase B | | | | |
| Aqua (Water) | 60.00 | 60.00 | 60.00 | 60.00 |
| Hydroxyethylcellulose | 1.50 | 1.50 | 1.50 | 1.50 |
| Total | 100.00 | 100.00 | 100.00 | 100.00 |

### Preparation:

Hydroxyethylcellulose is added to the water of phase B with vigorous stirring. The addition must take place sufficiently slowly that the particles are able to separate and their surfaces are individually wetted, but should also be sufficiently fast to minimise the viscosity of the aqueous phase. Dihydroxyacetone is added to the water of phase A, and the remaining ingredients are added with stirring. Phases A and B are combined and homogenised.

### Example 4

### Skin-Care Creams (O/W)

Preparation: phase A and phase B are heated to 80° C. Phase B is slowly stirred into phase A, and the mixture is subsequently homogenised. The mixture is allowed to cool with stirring, and phase C and subsequently phase D are added at 40° C.

| | | | ST-08-18 | ST-08-19 | ST-08-20 |
|---|---|---|---|---|---|
| Ingredient | SK-06-58 | ST-08-17 [%] | [%] | [%] | [%] |
| Phase A | | | | | |
| Tego Care 150 | 8.00 | 8.00 | 8.00 | 8.00 | 8.00 |
| Cetearyl Alcohol | 1.50 | 1.50 | 1.50 | 1.50 | 1.50 |
| Cetearyl | | 5.50 | 5.50 | 5.50 | 5.50 |
| Ethylhexanoate | | | | | |
| Cetearyl Octanoate | 6.50 | | | | |
| Miglyol 812N | 6.50 | 5.50 | 5.50 | 5.50 | 5.50 |
| Stearoxy | 1.20 | 1.00 | 1.00 | 1.00 | 1.00 |
| Dimethicone | | | | | |
| Dimethicone | 0.50 | 0.50 | 0.50 | 0.50 | 0.50 |
| Tocopheryl Acetate | 0.50 | 0.50 | 0.50 | 0.50 | 0.50 |
| Propylparaben | 0.05 | 0.05 | 0.05 | 0.05 | 0.05 |
| Phase B | | | | | |
| Ectoin | 0.50 | 0.30 | 0.30 | 0.30 | 0.30 |
| Propylene Glycol | 3.00 | 3.00 | 3.00 | 3.00 | 3.00 |
| Methylparaben | 0.15 | 0.15 | 0.15 | 0.15 | 0.15 |
| Aqua (Water) | 64.40 | 63.80 | 63.75 | 63.65 | 63.45 |
| Phase C | | | | | |
| Dihydroxyacetone | 1.00 | 0.10 | 0.15 | 0.25 | 0.45 |
| Probiol L 05018 | 1.00 | | | | |
| (empty liposomes) | | | | | |
| Aqua (Water) | 5.00 | 10.00 | 10.00 | 10.00 | 10.00 |
| Phase D | | | | | |
| Perfume | 0.20 | 0.10 | 0.10 | 0.10 | 0.10 |
| Total | 100.00 | 100.00 | 100.00 | 100.00 | 100.00 |
| | | | ST-08-23 | ST-08-24 | ST-08-25 |
| Ingredient | ST-08-21 | ST-08-22 [%] | [%] | [%] | [%] |
| Phase A | | | | | |
| Octocrylene | | | | 2.00 | 2.00 |
| Butyl Methoxy- | | | | 2.00 | 2.00 |
| dibenzoylmethane | | | | | |
| Tego Care 150 | 8.00 | 8.00 | 8.00 | 8.00 | 8.00 |
| Cetearyl Alcohol | 1.50 | 1.50 | 1.50 | 1.50 | 1.50 |
| Cetearyl | 5.50 | 5.50 | 5.50 | 5.50 | 5.50 |
| Ethylhexanoate | | | | | |
| Miglyol 812N | 5.50 | 5.50 | 5.50 | 5.50 | 5.50 |
| Stearoxy | 1.00 | 1.00 | 1.00 | 1.00 | 1.00 |
| Dimethicone | | | | | |
| Dimethicone | 0.50 | 0.50 | 0.50 | 0.50 | 0.50 |
| Tocopheryl Acetate | 0.50 | 0.50 | 0.50 | 0.50 | 0.50 |
| Propylparaben | 0.05 | 0.05 | 0.05 | 0.05 | 0.05 |
| Phase B | | | | | |
| Ectoin | 0.30 | 0.30 | 0.30 | 0.30 | 0.30 |
| Propylene Glycol | 3.00 | 3.00 | 3.00 | 3.00 | 3.00 |
| Ethoxydiglycol | | 1.00 | 1.00 | 1.00 | 1.00 |
| Methylparaben | 0.15 | 0.15 | 0.15 | 0.15 | 0.15 |
| Aqua (Water) | 63.40 | 62.40 | 62.10 | 58.40 | 58.10 |
| Phase C | | | | | |
| Dihydroxyacetone | 0.50 | 0.50 | 0.50 | 0.50 | 0.80 |
| Erythrulose | | | 0.30 | | |
| Aqua (Water) | | 10.00 | 10.00 | 10.00 | 10.00 |
| Phase D | | | | | |
| Perfume | | 0.10 | 0.10 | 0.10 | 0.10 |
| Total | | 100.00 | 100.00 | 100.00 | 100.00 |

| Trade name | INCI |
|---|---|
| Tego Care 150 | Glyceryl stearate, Steareth-25, |
| | Ceteth-20, Stearyl Alcohol |
| Miglyol 812 | Caprylic/Capric TriglycerideN |
| Probiol L 05018 (empty liposome) | Aqua, Alcohol denat., Lecithin, |
| | Glycerin, Disodium phosphate |

### Example 5

### Analysis of Colouring Intensity and Homogeneity on Repetitive Use of Skin Cream SK-06-58 from Example 4 Comprising 1% of DHA

The investigation was carried out on 6 test subjects. During a pretreatment time of 7 days and during application, no other cosmetic formulations were allowed to be used. Exposure to UV light and artificial UV radiation was forbidden.

Test formulation SK-06-58, comprising 1% of DHA, was tested against a standard formulation comprising 5% of DHA. The formulations were applied in an amount of 2 μl/cm², with SK-06-58 being applied to five areas of the forearm and the comparative formulation comprising 5% to two areas of the same arm. The study lasted 5 days. SK-06-58 was used once every day, with all five areas of the forearm being treated the first time, four areas on the 2nd day, and so on. The comparative formulation was applied once on the first day and to the second area on the first and fifth days. The skin colour was assessed visually with respect to intensity and homogeneity, and L*a*b* values (Commission Internationale de l'Eclairage (CIE Publication, 1986)) were determined using a CR300 Chromameter® (Minolta, Osaka, Japan).

### Results:

### 1. Visual Results of Skin Colouring and Homogeneity

| Degree of | Intensity of | Degree of | |
|---|---|---|---|
| colouring | colouring | homogeneity | Homogeneity |
| 0 | no tanning | | |
| 1-1.5 | slight tanning | 1-1.5 | weak |
| 2-2.5 | moderate tanning | 2-2.5 | moderate |
| 3-3.5 | strong tanning | 3-3.5 | strong |
| 4 | very strong tanning | 4 | very strong |

The tanning values on use of SK-06-58 increased from 0.75±0.42 (1×) to 1.25±0.42 (2×) to 1.67±0.41 (3×) to 2.08±0.49 (4×) to 2.67±0.52 (5×). The value achieved by the comparative formulation is 2.17±0.42 six hours after the first application and 1.17±0.68 after 5 days.

The results therefore confirm the advantages of the method according to the invention.

For SK-06-58, the following homogeneity values were achieved: from 2.50±0.63 (1×) to 2.83±0.41 (2×) to 3.08±0.58 (3×) to 3.33±0.52 (4×) to 3.67±0.41 (5×). The value achieved by the comparative formulation is 3.75±0.42 six hours after the first application and 2.17±0.68 after 5 days.

### 2. Measurement Results Using a CR300 Chromameter® (Minolta, Osaka, Japan)

The b* values and L*a*b* values are listed below:

| | b* values | | | | |
|---|---|---|---|---|---|
| | (1x) | (2x) | (3x) | (4x) | (5x) |
| SK-06-58 | 0.87 ± 0.89 | 1.13 ± 1.35 | 2.18 ± 1.84 | 3.31 ± 1.96 | 3.52 ± 2.40 |
| 5% of DMA | 2.49 ± 0.94 | | | | 1.49 ± 2.02 |
| | L*a*b* | | | | |
| | (1x) | (2x) | (3x) | (4x) | (5x) |
| SK-06-58 | 2.88 ± 1.39 | 3.49 ± 2.17 | 4.06 ± 2.33 | 5.54 ± 3.25 | 6.05 ± 3.23 |
| 5% of DMA | 4.40 ± 1.63 | | | | 3.02 ± 2.61 |

These results therefore also confirm the method according to the invention.

1. Method for tanning the human body, characterised in that at least one formulation comprising 0.1 to 1% by weight of at least one self-tanning agent substance is applied repeatedly, but at least once daily, to the human body or parts of the human body.

2. Method for avoiding application edges during tanning of the human body, characterised in that at least one formulation comprising 0.1 to 1% by weight of at least one self-tanning agent substance is applied repeatedly, but at least once daily, to the human body or parts of the human body.

3. Method according to claim 1, characterised in that the at least one formulation comprises more than 0.1% by weight and less than 0.5% by weight of self-tanning agent substance.

4. Method according to claim 1, characterised in that the at least one self-tanning agent substance is essentially dihydroxyacetone or erythrulose or a mixture thereof.

5. Method according to claim 1, characterised in that the at least one formulation is applied twice or three times daily, preferably twice daily.

6. Method according to claim 1, characterised in that a day-care formulation comprising 0.1 to 1% by weight of at least one self-tanning agent substance is applied in the morning and a night-care formulation comprising 0.1 to 1% by weight of at least one self-tanning agent substance is applied in the evening.

7. Cosmetic formulation comprising at least one self-tanning substance, characterised in that the formulation comprises more than 0.1% by weight and less than 0.5% by weight of the at least one self-tanning agent substance.

8. Cosmetic formulation according to claim 7, characterised in that the at least one self-tanning agent substance is essentially dihydroxyacetone or erythrulose or a mixture thereof.

9. Cosmetic formulation according to claim 1, characterised in that the formulation comprises 0.1 to 1% by weight of the at least one self-tanning agent substance.

10. Cosmetic formulation according to claim 1, characterised in that the formulation comprises one or more UV filters.

11. Cosmetic formulation according to claim 1, characterised in that the formulation comprises glycerol and/or propylene glycol as hydrophilic solvent, preferably in a concentration of 0.1 to 20% by weight.

12. Cosmetic formulation according to claim 1, comprising at least one self-tanning substance, characterised in that the formulation is a liquid, creamy, milky and/or gelatinous day-care product.
